(19) United States
(12) Patent Application Publication
(10) Pub. No.: US 2005/0009902 A1
(43) Pub. Date: Jan. 13, 2005

(54) Remedies for pruritus

(76) Inventors: Suguru Miyaji, Ikoma (JP); Masatomo Kato, Ikoma (JP)

Correspondence Address: FRISHAUF, HOLTZ, GOODMAN & CHICK, PC, 767 THIRD AVENUE, 25TH FLOOR, NEW YORK, NY, 10017-2023, US

(21) Appl. No.: 10/493,563

(22) PCT Filed: Oct. 22, 2002
(86) PCT No.: PCT/JP02/10912

(30) Foreign Application Priority Data
Oct. 22, 2001 (JP) 2001-323218

(51) Int. Cl. A61K 31/353; A61K 31/16

(52) U.S. Cl. 514/453; 514/625

(57) ABSTRACT

The invention aims to find out a novel pharmacological effect (medical use) of cannabinoid. Since cannabinoid agonists such as palmidrol and anandamide have an excellent antipruritic effect, they are useful as an agent for treating any types of pruritus such as ocular pruritus, skin pruritus and systemic pruritus.

### TECHNICAL FIELD

The present invention relates to a therapeutic agent for pruritus containing a cannabinoid agonist as an active ingredient.

### BACKGROUND ART

With respect to pruritus, a pruritus receptor present in the dermoepidermal junction of the skin or the mucous membrane is stimulated with a mediator (pruritus-inducing substance), and this stimulation is transmitted to the central nerve and felt as pruritus. As a mediator that induces pruritus, for example, histamine, kinin, bile acid salts, substance P and prostaglandin have been widely known. A mediator released from mast cells, such as histamine, is presumably involved in pruritus caused by allergic factors, and an antihistaminic agent has been widely used to reduce the itching.

As pruritus, for example, ocular pruritus, skin pruritus and systemic pruritus which are caused in humans or animals are known. Examples of diseases accompanied with ocular pruritus include allergic conjunctivitis, vernal keratoconjunctivitis, atopic keratoconjunctivitis, infectious keratoconjunctivitis and blepharitis. Pruritus is also caused by ophthalmic operation such as cataract operation. Allergic conjunctivitis is known to be triggered by various causes such as pollen, dust, mites, molds, pet's hair, contact lens and cosmetics. Eyes, eyelids and edges of eyelids become itchy. Scratching eyes leads to hyperemia at conjunctiva, and the papilla of the conjunctiva becomes red and grows. In a serious case, the lesion appears in the cornea or the sclera, which leads to a more serious disease, vernal keratoconjunctivitis.

By the way, it is known that cannabinoid is a generic term of an active ingredient of cannabis and acts on the central nerve. As the cannabinoid agonist, for example, peripheral-type cannabinoid receptor agonists such as indometacin morpholinylamide and palmidrol, and non-selective-type cannabinoid receptor agonists such as anandamide and tetrahydrocannabinoid are known. Further, 2-oxoquinoline derivatives having immunomodulating activity, antiinflammatory activity and antiallergic activity as described in JP-A-2000-256323 are known to be an excellent cannabinoid agonist.

JP-T-11-500411 (the term “JP-T” as used herein means a published Japanese translation of a PCT patent application) describes that cannabinoid is useful for treatment of diseases associated with modulation of a peripheral cannabinoid receptor and it can specifically be used in diseases accompanied with degeneracy of pain sensation, multiple sclerosis, diseases accompanied with change of intraocular pressure, chronic degeneration diseases such as chronic respiratory disorders, senile dementia and Alzheimer. JP-A-5-345722 describes the invention concerning pharmaceutical compositions comprising N-acyl derivatives of an aminoalcohol such as N-palmitoyl-ethanolamide (palmidrol) which is a cannabinoid agonist. These pharmaceutical compositions are used as an agent for treating autoimmune diseases such as atopic dermatitis, dermatomyositis, sympathetic ophthalmia, autoimmune uveitis, uveal retinopathy and keratoconjunctivitis sicca. This document describes that the administration of N-acyl derivatives of aminoalcohol inhibits the mast cell degranulation reaction to prevent liberation of a mediator such as histamine from mast cells, that is, N-acyl derivatives of an aminoalcohol act on mast cells to inhibit autoimmune diseases.

The cannabinoid agonist is known to have various pharmacological effects as a drug, and it is an interesting subject to further find out a new pharmacological effect.

### DISCLOSURE OF THE INVENTION

The present inventors have performed pharmacological tests, and have consequently found that cannabinoid agonists such as palmidrol, indometacin morpholinylamide, anandamide and 2-oxoquinoline derivatives described in JP-A-2000-256323 exhibit excellent antipruritic activity to ocular pruritus. Further, from the results of pruritus inhibition tests using histamine-induced models and platelet activating factor-induced models, it has been clarified that the cannabinoid agonist of the invention directly acts on the peripheral nerve terminal to inhibit transmission of a pruritus signal in nerve cells.

The invention is a therapeutic agent for pruritus comprising, as an active ingredient, a cannabinoid agonist such as palmidrol, indometacin morpholinylamide, anandamide or 2-oxoquinoline derivatives. 2-oxoquinolie derivatives refer to compounds described in JP-A-2000-256323.

The cannabinoid agonist of the present invention brings forth the effect of treating or inhibiting pruritus caused in humans and animals. Preferably, it is used as an agent for treating ocular pruritus. The pruritus may be pruritus in which mast cells are not involved, for example, ocular pruritus in which mast cells are not involved.

Examples of diseases accompanied with ocular pruritus include allergic conjunctivitis, vernal keratoconjunctivitis, atopic keratoconjunctivitis, infectious keratoconjunctivitis and blepharitis. Pruritus is also caused by ophthalmic operation such as cataract operation.

The cannabinoid agonist of the invention may be either a peripheral-type cannabinoid receptor agonist or a non-selective-type cannabinoid receptor agonist. Examples of the peripheral-type cannabinoid receptor agonist include indometacin morpholinylamide, N-acyl derivatives of an aminoalcohol such as palmidrol as described in JP-A-5-345722, 2-oxoquinoline derivatives described in JP-A-2000-256323, 2-imino-1,3-thiazine derivatives described in WO 01/19807, pyridone derivatives described in WO 02/53543, 3,4-dihydroisoquinoline derivatives described in WO02/10135, pyrazole derivatives described in JP-T-2001-516361 and the like. Palmidrol, indometacin morpholinylamide and 2-oxoquinoline derivatives are preferably used. Specific examples of 2-oxoquinoline derivatives include N-(benzo[d][1,3]dioxol-5-ylmethyl)-7-methoxy-2-oxo-8-pentyloxy-1,2-dihydroquinoline-3-carboxamide, 8-butoxy-7-methoxy-2-oxo-1,2-dihydroquinoline-3-carboxylic acid and the like. Examples of the non-selective-type cannabinoid receptor agonist include anandamide, tetrahydrocannabinoid and the like. Anandamide is preferably used.

The therapeutic agent for pruritus according to the present invention, as is apparent from the results of the pruritus inhibition tests using histamine-induced models and platelet activating factor-induced models in the pharmacological tests described later, directly acts on peripheral nerve terminals to inhibit transmission of the pruritus signal in nerve cells, so that it can exhibits the excellent antipruritic effect to pruritus caused by any factors.

The therapeutic agent for pruritus of the invention can be formed into preparations by adding pharmaceutically acceptable additives, as required, according to the technique which is widely used to obtain single preparations or mixed preparations. Moreover, the therapeutic agent for pruritus of the invention can be administered either parenterally or orally.

Examples of the dosage form in case of a therapeutic agent for ocular pruritus include eye drops, eye ointments, tablets and the like. The preferable dosage form is eye drops or eye ointments. These can be prepared through a technique which is widely used. For example, an eye drop can be prepared by mixing, as additives, an isotonic agent, a buffer, a pH adjustor, a solubilizer, a thickener, a stabilizer, a preservative and the like as required. Further, a stable eye drop can be obtained by adding a pH adjustor, a thickener, a dispersant and the like to suspend the agents.

Examples of the isotonic agent can include glycerin, propylene glycol, sodium chloride, potassium chloride, sorbitol, mannitol and the like.

Examples of the buffer include phosphoric acid, phosphate, citric acid, acetic acid, e-aminocaproic acid, tromethamol and the like.

Examples of the pH adjustor include hydrochloric acid, citric acid, phosphoric acid, acetic acid, sodium hydroxide, potassium hydroxide, boric acid, borax, sodium carbonate, sodium hydrogencarbonate and the like.

Examples of the solubilizer include polysorbate 80, polyoxyethylene hydrogenated castor oil 60, macrogol 4000 and the like.

Examples of the thickener and the dispersant include cellulosic polymers such as hydroxypropylmethyl cellulose and hydroxypropyl cellulose, polyvinyl alcohol, polyvinylpyrrolidone and the like.

Examples of the stabilizer include edetic acid, sodium edetate and the like.

Examples of the preservative (antiseptic) include sorbic acid, potassium sorbate, benzalkonium chloride, benzethonium chloride, methyl p-oxybenzoate, propyl p-oxybenzoate, chlorobutanol and the like which are widely used. These preservatives may also be used in combination.

In the eye drops containing the therapeutic agent for pruritus of the invention, the pH is preferably in the range between 4.0 and 8.0, and the osmotic pressure rate is preferably at approximately 1.0.

The invention also relates to a method for treating pruritus, which comprises administering to a patient a therapeutically effective amount of cannabinoid agonist.

The dose in case of a therapeutic agent for ocular pruritus can properly be selected depending on the symptoms, the age, the dosage form and the like. With respect to the eye drops, it may be instilled from once to several times a day at a concentration of from 0.001 to 10% (w/v), preferably from 0.01 to 1% (w/v).

Best Mode for Carrying Out the Invention

Preparation Examples and the results of pharmacological tests are described below. However, these examples are for understanding well the invention, and do not limit the scope of the invention.

### PREPARATION EXAMPLES

Typical Preparation Examples used in the invention are described below.

1. Eye Drop

An eye drop according to the following formulation is prepared by a method which is widely used.

Formulation 1

per 100 ml

| | |
|---|---|
| palmidrol | 500 mg |
| conc. glycerin | 500 mg |
| polysorbate 80 | 200 mg |
| sodium dihydrogenphosphate dihydrate | q.s. |
| 1N sodium hydroxide | q.s. |
| hydrochloric acid | q.s. |
| sterile purified water | q.s. |

An eye drop containing palmidrol at a concentration of 10 mg, 50 mg, 100 mg or 1,000 mg per 100 ml can be prepared in the same manner as in Formulation 1. Indometacin morpholinylamide, anandamide or 2-oxoquinoline derivatives can be used instead of palmidrol.

2. Eye Ointment

An eye ointment according to the following formulation is prepared by a method which is widely used.

Formulation 2

per 100 ml

| | |
|---|---|
| palmidrol | 300 mg |
| liquid paraffin | 10 g |
| white vaseline | q.s. |

Eye ointments at various concentrations of palmidrol can be prepared in the same manner as in Formulation 2 by properly changing the concentration of palmidrol. Indometacin morpholinylamide, anandamide or 2-oxoquinoline derivatives can be used instead of palmidrol.

(Pharmacological Tests)

The ocular pruritus inhibition activity of the cannabinoid agonist was examined using histamine-induced models, allergic conjunctivitis models and platelet activating factor-induced models.

(1) Ocular Pruritus Inhibition Activity to Histamine-Induced Models

<Experimental Method>

Palmidrol was suspended in a physiological saline at a concentration of 0.1% or 0.5%(w/v), and the resulting suspension was instilled into both eyes of a 5-week-old male Hartley guinea pig in 10 μL/one eye. After 10 minutes, the palmidrol suspension at the same concentration was also instilled thereinto (twice in total). A physiological saline was used as a control.

After 5 minutes from the second instilling of palmidrol, a physiological saline containing 1.0% (w/v) histamine was instilled into both eyes of the guinea pig in 10 μL/one eye to induce eye scratching action.

The behavior of the guinea pig after administering histamine was videotaped, and the ocular pruritus was evaluated on each eye by counting actions of scratching the eye with the hind foot. The average value of the number of eye scratchings for 30 minutes after administering histamine and the eye scratching inhibition rate are shown in Table 1. In each example, 12 eyes are used.

Eye scratching inhibition rate=100−(number of eye scratchings with test compound)/(number of eye scratchings with control)×100

TABLE 1

| | Number of eye | Eye scratching |
|---|---|---|
| | scratchings (times) | inhibition rate (%) |
| Control | 22.8 | — |
| Palmidrol (0.1%) | 8.5 | 62.8 |
| Palmidrol (0.5%) | 3.8 | 83.2 |

(Experimental Results)

The number of eye scratchings of guinea pigs into whose eyes palmidrol had been instilled was markedly decreased in comparison with the control. Thus, palmidrol was confirmed to have the ocular pruritus inhibition effect. The extent thereof depends on the concentration of palmidrol.

(2) Ocular Pruritus Inhibition Activity to Allergic Conjunctivitis Models

(Experimental Method)

Aluminum hydroxide gel-adsorbed ovalbumin (20 μg/mL) was dissolved in a physiological saline, and the resulting solution was subconjunctivally injected into both eyes of a 4-week-old male Hartley guinea pig in 100-μL portions for active sensitization. On days 14, 16, 18, 21, 23 and 25 after the sensitization, a physiological saline containing 2.5% (w/v) ovalbumin was instilled into both eyes in 10 μL/one eye to induce allergic conjunctivitis. A physiological saline was used as a control.

Five minutes and 15 minutes (twice in total) before the instilling of ovalbumin on days 16, 18, 21, 23 and 25 after the sensitization, a suspension of 0.1% (w/v) palmidrol in a physiological saline was instilled into both eyes of the guinea pig in 10 μL/one eye.

The behavior of the guinea pig for 30 minutes after the instilling of ovalbumin on days 21, 23 and 25 after the sensitization were videotaped, and the ocular pruritus was evaluated by counting the number of eye scratchings in case of instilling palmidrol and a control. The eye scratching inhibition rate (average value) to the control is shown in Table 2. In each example, 12 eyes are used.

TABLE 2

Eye scratching inhibition rate (%)

| | Day 21 after | Day 23 after | Day 25 after |
|---|---|---|---|
| | sensitization | sensitization | sensitization |
| Palmidrol | 41.5 | 52.3 | 51.7 |
| (0.1%) | | | |

(Experimental Results)

As is apparent from Table 2, since the eye scratching action of the guinea pig into whose eyes palmidrol had been instilled was significantly inhibited in comparison with the control, palmidrol was confirmed to have the ocular pruritus inhibition effect.

(3) Ocular Pruritus Inhibition Activity to Platelet Activating Factor-Induced Models

The ocular pruritus inhibition activity to platelet activating factor (PAF)-induced models was examined using anandamide (test compound A), indometacin morpholinylamide (test compound B) and N-(benzo[d][1,3]dioxol-5-ylmethyl)-7-methoxy-2-oxo-8-penty loxy-1,2-dihydroquinoline-3-carboxamide (test compound C).

(Experimental Method)

Test compound A (50 mg/ml in EtOH) was suspended in a physiological saline at a concentration of 0.01% (w/v). Test compound B was suspended in a 1.0% Tween 80/physiological saline at a concentration of 0.001% or 0.01% (w/v). Test compound C was suspended in a 5.0% Tween 80/physiolosical saline at a concentration of 0.0001%, 0.001% or 0.01% (w/v). In this manner, the test compound suspensions were prepared.

With respect to test compounds A and B, each of the test compound suspensions was instilled into both eyes of a 5-week-old male Hartley guinea pig in 10 μL/one eye. Ten minutes later, each of the test compound suspensions was instilled thereinto at the same concentration (twice in total). With respect to test compound C, the test compound suspension was instilled into both eyes of the 5-week-old male Hartley guinea pig in 10 μL/one eye.

Subsequently, with respect to test compounds A and B, a physiological saline containing 0.1% (w/v) of the platelet activating factor was instilled in 10 μL/one eye into both eyes of the guinea pig after 5 minutes from the second instilling of the respective test compound suspensions. With respect to test compound C, the same physiological saline was instilled in 10 μL/one eye into both eyes of the guinea pig after 15 minutes from the instilling of the test compound suspension. In this manner, the eye scratching action was induced. As controls of test compounds A, B and C, a 2.0% ethanol/physiological saline, a physiological saline and a 5.0% Tween 80/physiological saline were used respectively.

The behavior of the guinea pig after instilling the platelet activating factor was videotaped, and the ocular pruritus was evaluated for each eye by counting the number of actions of scratching the eye with the hind foot. The average value of the eye scratching inhibition rate for 30 minutes after instilling the platelet activating factor is shown in Table 3. In each example, 8 eyes are used.

TABLE 3

| | Eye scratching |
|---|---|
| | inhibition rate (%) |
| Test compound A (0.01%) | 40.9 |
| Test compound B (0.001%) | 38.7 |
| Test compound B (0.01%) | 52.9 |
| Test Compound C (0.0001%) | 22.7 |
| Test Compound C (0.001%) | 34.3 |
| Test Compound C (0.01%) | 44.2 |

(Experimental Results)

From Table 3, it was confirmed that since the number of eye scratchings of the guinea pigs into whose eyes test compounds A, B and C had been instilled was markedly decreased in comparison with the controls, the respective test compounds all had the ocular pruritus inhibition effect. The extent thereof depends approximately on the concentrations of the respective test compound suspensions.

As is clear from the results of the pharmacological tests, palmidrol, indometacin morpholinylamide, anandamide and 2-oxoquinoline derivatives exhibit the excellent antipruritic effect to the ocular pruritus.

### INDUSTRIAL APPLICABILITY

The cannabinoid agonist of the invention directly acts on the peripheral nerve terminal to inhibit transmission of a pruritus signal in nerve cells. Accordingly, it can exhibit the excellent antipruritic effect to pruritus caused by any factors.

1. A therapeutic agent for pruritus comprising an effective amount as a cannabinoid agonist of a cannabinoid agonist as an active ingredient in admixture with pharmaceutically acceptable additives.

2. The therapeutic agent for pruritus according to claim 1, wherein the cannabinoid agonist is a peripheral-type cannabinoid receptor agonist or a non-selective-type cannabinoid receptor agonist.

3. The therapeutic agent for pruritus according to claim 1, wherein the cannabinoid agonist is a palmidrol derivative.

4. (Cancelled)

5. (Cancelled)

6. The therapeutic agent for pruritus according to claim 1, in the form of an eye drop or an eye ointment.

7. (Cancelled)

8. (Cancelled)

9. A method for treating pruritus, which comprises administering to a patient a therapeutically effective amount of a cannabinoid agonist.

10. The method for treating pruritus according to claim 9, wherein the cannabinoid agonist is a peripheral-type cannabinoid receptor agonist or a non-selective cannabinoid receptor agonist.

11. The method for treating pruritus according to claim 9, wherein the cannabinoid agonist is a palmidrol derivative.

12. The method for treating pruritus according to any one of claims 9 to 11, wherein the pruritus is ocular pruritus.

13. The method for treating pruritus according to claim 12, wherein the ocular pruritus is pruritus caused by allergic conjunctivitis, vernal keratoconjunctivitis, atopic keratoconjunctivitis, infectious keratoconjunctivitis, blepharitis and ophthalmic operation.

14. The method for treating pruritus according to claim 12, wherein the dosage form is an eye drop or an eye ointment.

15. The method for treating pruritus according to any one of claims 9 to 11, wherein the pruritus is pruritus in which mast cells are not involved.

16. The method for treating pruritus according to claim 12, wherein the pruritus is ocular pruritus in which mast cells are not involved.

17-24. (Cancelled)

25. The therapeutic agent for pruritus according to claim 6, wherein the cannabinoid agonist is present in an amount of 0.001 to 10% w/v of the therapeutic agent.

26. The therapeutic agent for pruritus according to claim 6, wherein the cannabinoid agonist is present in an amount of 0.01 to 1% w/v of the therapeutic agent.

27. The therapeutic agent for pruritus according to claim 1, wherein the cannabinoid agonist is a indometacin morpholinylamide derivative.

28. The therapeutic agent for pruritus according to claim 1, wherein the cannabinoid agonist is a anandamide derivative.

29. The therapeutic agent for pruritus according to claim 1, wherein the cannabinoid agonist is a 2-oxoquinoline derivative.

30. The method for treating pruritus according to claim 9, wherein the cannabinoid agonist is a indometacin morpholinylamide derivative.

31. The method for treating pruritus according to claim 9, wherein the cannabinoid agonist is a anandamide derivative.

32. The method for treating pruritus according to claim 9, wherein the cannabinoid agonist is a 2-oxoquinoline derivative.
